(19) United States
(12) Patent Application Publication
(10) Pub. No.: US 2009/0291462 A1
(43) Pub. Date: Nov. 26, 2009

(54) Detection or Quantification of Aggrecan and its Fragments

(75) Inventors: Eren Ufuk Sumer, Frederiksberg (DK); Per Qvist, Klampenborg (DK)

Correspondence Address: EDWARDS ANGELL PALMER & DODGE LLP, P.O. BOX 55874, BOSTON, MA, 02205, US

(73) Assignee: Immunodiagnostic Systems Nordic A/S, Herlev (DK)

(21) Appl. No.: 12/083,900

(22) PCT Filed: Oct. 17, 2006
(86) PCT No.: PCT/EP2006/067520

§ 371 Date: Feb. 11, 2009

(30) Foreign Application Priority Data
Oct. 20, 2005 (GB) 0521392.1
May 5, 2006 (GB) 0608947.8

(51) Int. Cl. G01N 33/53; C07K 16/00; C12N 5/00

(52) U.S. Cl. 435/7.94; 435/7.1; 530/387.1; 435/325

(57) ABSTRACT

An immunoassay for aggrecan and/or aggrecan derived fragments comprises contacting a sample with an immunological binding partner which has specific binding affinity for the G2 domain of aggrecan at least when bearing keratan sulphate chains, and determining the existence or amount of specific binding of the immunological binding partner, which may be conducted as a sandwich assay using a first antibody that binds an N-terminal amino acid sequence comprising FFGVG . . . and a second antibody that binds an N-terminal amino acid sequence comprising ARGS.

The present invention relates to assays for the detection and/or quantification of aggrecan and fragments thereof by using antibodies or other immunological specific binding partners recognising the G2 domain.

Aggrecan is synthesised by the chondrocytes (Archer and Francis, 2003) and is a major constituent of the articular cartilage of the joints, where it is organised with type II collagen and other matrix molecules (Hardingham and Fosang, 1995).

Aggrecan is heavily glycosylated and comprises more than 2000 amino-acid residues. Aggrecan is structurally organized in three distinct domains: G1, G2 and G3 (Fig. A). Interspaced between the G2 and G3 domain, and to a lesser extent between the G1 and G2 domains are long stretches of heavily glycosylated regions, containing the negatively charged chondroitin sulphate and keratan sulphate oligosaccharide structures (Hardingham & Fosang 1995).

Osteoarthritis is characterised by an irreversible destruction of articular cartilage. Chondrocytes attempt to repair the degenerating cartilage by synthesising new matrix constituents, including aggrecan (Garnero et al., 2000).

There is a need for biochemical markers, which can provide information on the metabolic processes in the cartilage. Biochemical markers have been described and measurement of aggrecan and its fragments has been reported.

The CS 846 test uses antibodies recognising the chondroitin sulfate sidechain bound to amino acid 846 between the G2 and the G3 domain of the aggrecan molecule (IBEX Pharmaceuticals Inc.). The FA-846 sandwich immunoassay, which is an adaptation of the CS 846 test for the quantification of fetal aggrecan, has also been described.

Other tests for aggrecan have been developed, e.g. “Aggrecan Proteoglycan” (Biosource, US). However, the specificity of the antibodies remains to be determined.

Other aggrecan assays target the glycosaminoglycan region of aggrecan, i.e. between the G2 and the G3 domain (Kongtawelert and Ghosh 1990; Ratcliffe et al., 1993).

Møller et al., have developed a competition ELISA for the core protein part of aggrecan, though not specifying the binding region of the antibody (Møller et al., 1994).

The antibody, 1-C-6 has been developed which binds to both the G1 and non-masked G2 domains (Fosang and Hardingham, 1991). The keratan sulphate side chains had to be removed using keratanase for reactivity with 1-C-6 with the G2 domain. Accordingly, the 1-C-6 antibody is not suitable for use in assays for aggrecan or aggrecan fragments in body fluids or body tissues.

Numerous proteolytic clevage-sites have been described for aggrecan (Fosang et al., 2000; Caterson et al., 2000), and a predominant site for the metalloproteinases (MMPs) is located in the intra-globular domain (IGD) between amino acid N³⁴¹ and F³⁴² (Fosang et al. 1996). A monoclonal antibody, i.e. AF28 (ATCC HB11671), that specifically binds the polypeptide neo-epitope containing the N-terminal sequence ³⁴²FFGVG . . . , has previously been developed (Fosang et al. 1995). The AF28 antibody has been used in competition ELISA for detection of aggrecan fragments in synovial fluid and human serum (Fosang et al., 1995). However, the use of this antibody, in combination with antibodies to the G2-domain has not been reported.

Aggrecan is referred to in a number of patent publications. Several of these refer to measurement of aggrecan or certain characteristic fragments of the protein with a diagnostic purpose to assess cartilage catabolism. U.S. Pat. No. 4,704,356 discloses that abnormal levels of keratan sulfate (KS) in the peripheral blood are indicative of abnormalities of cartilage or cartilage-like tissues. Elevated levels of KS in the peripheral blood are described as being indicative of osteoarthritis. Interestingly absence of KS as well as very elevated levels of KS in the peripheral blood were found to be indicative of muscular dystrophy and related disorders. The technique used for quantification of KS in the peripheral blood was an immunoassay using a monoclonal antibody.

U.S. Pat. No. 5,935,796 describes other diagnostic methods and compositions relating to the proteoglycan proteins of cartilage breakdown. Methods are described for early diagnosis, monitoring and treatment of osteoarthritis using monoclonal antibodies which specifically recognize antigenic determinants on atypical chondroitin sulfate (CS)/dermatan sulfate glycosaminoglycan chains in body tissues and fluids, that originate from articular cartilage aggrecan.

U.S. Pat. No. 4,778,768 describes methods for monitoring the progressive destruction of articular cartilage in joints, and more specifically for determining changes occurring in articular cartilage. The method involves (a) quantifying proteoglycan monomer and/or antigenic fragments thereof in a synovial fluid sample and (b) correlating the values thus obtained with progressive destructions in the articular cartilage appertaining to that sample fluid. The proteoglycan fragments were measured by an immunoassay employing an antibody specific to proteoglycan monomers. The assay described in this patent appears to be identical with the polyclonal HABr ELISA described above.

U.S. Pat. No. 5,948,692 describes an assay, which uses a size separation method for dividing glycans having avidity for hyaluronic acid (HA) from proteoglycans not having such avidity. The assay measures the HA binding proteoglycans, such as aggrecan. This is said to enable the biochemical diagnosis of joint diseases in the field of orthopedics as well as RA, OA and other joint diseases. The method can it is said be utilized also for discriminating normal joints from pathologic joints, for providing a prognostic measure of disease progression and for monitoring the effects of therapeutic interventions.

U.S. Pat. No. 5,427,954 describes the use of an immunoassay for measurement of aggrecan containing a neo-epitope ARGSVI. This is one of a number of disclosures describing the diagnostic utility of neo-epitopes generated by specific proteolysis of aggrecan mediated by proteases involved in the pathological processes of joint diseases.

U.S. Pat. No. 5,387,504 describes the neo-epitope VDIPEN released by the action of stromelysin at the site N₃₄₁-F₃₄₂ and an RIA assay employing a monoclonal antibody specific for this epitope. More generally the use of monospecific antibodies specific for fragments of aggrecan, generated by specific stromelysin cleavage are described. Elevations of stromelysin occur in osteoarthritis, rheumatoid arthritis, atherosclerotic lesions, gout, inflammatory bowel disease (IBD), idiopathic pulmonary fibrosis (IPF), certain cancers, joint injuries, and numerous inflammatory diseases. At present the clinical value of assays specific for these aggrecan ‘neo-epitopes’ is not well known, and it is not established whether these fragments are released into circulation in significant amounts and how they are catabolised.

U.S. Pat. No. 5,935,796 relates to methods and compositions for early diagnosis, monitoring and treatment of cartilage degenerative conditions, using an antibody which recognizes a peptide comprising the sequence FFGVG generated by cleavage of cartilage aggrecan at the site N₃₄₁-F₃₄₂. This epitope is the ‘other end’ of the VDIPEN epitope released by the action of stromelysin on aggrecan. It is suggested to provide a sandwich assay to improve the sensitivity of detection of FFGVG fragments of aggrecan, more specifically a sandwich assay combining AF-28 with an anti-keratan sulphated antibody such as 5-D-4. However, until now, attempts to make satisfactory sandwich assays using AF-28 as one of two antibodies have been unsuccessful in practice.

U.S. Pat. No. 5,185,245 describes an immunoassay for detection of proteoglycans in synovial fluid and methods of monitoring treatment of diseases characterised by breakdown of proteoglycans. A test sample of synovial fluid is quantified by an immunoassay employing antibodies specifically recognizing proteoglycan, where the antibodies are immobilized on a solid support. Bound proteoglycan is then contacted with a second specific antibody, which is labeled with a detection reagent (i.e. peroxidase). Both antibodies have affinity to the glycosaminoglycan (GAG/CS) moieties on the proteoglycan.

U.S. Pat. No. 5,354,662 and U.S. Pat. No. 5,217,903 describe generally the measurement of ‘tissue breakdown products’ in body fluids based on quantification of a connective tissue or muscle tissue breakdown product in a body fluid from an animal by using a standard comprising the breakdown product having a radioactive label. The standard should have a known specific activity and thus combining the standard and a sample of the body fluid, the specific radioactivity measured in a RIA/IRMA type assay can be used as a measure of the quantity of the breakdown product in the sample. Also described are methods for assessing, in a body fluid from an animal, the condition of a selected connective tissue or a muscle tissue in an animal, and for assessing a disease process that includes destruction of a specified connective tissue component or muscle tissue, and for assessing the efficacy of a therapy for treatment of such a disease process, include the steps of the method for determining the quantity of a tissue breakdown product.

None of these above mentioned methods employ antibodies recognising epitopes located on the G2-domain of aggrecan which are able to be bound prior to keratanase treatment, or discuss the use of such antibodies.

The present invention is based on the discovery, that antibodies to the G2 domain of aggrecan provide superior utility in immunoassays for the detection or quantification of aggrecan.

It was discovered, that antibodies targeting epitopes of the G2-domain that are not natively masked provide advantages in immunoassays for measurements of aggrecan.

Accordingly, the present invention provides in a first aspect, an immunoassay method for the detection or quantitative determination of aggrecan and/or aggrecan derived fragments in a sample comprising contacting the sample with an immunological binding partner which has specific binding affinity for the G2 domain of aggrecan bearing keratan sulphate chains, and determining the existence or amount of specific binding of the immunological binding partner.

Optionally, said immunological binding partner has specific binding affinity for the G2 domain of aggrecan, both when said G2 domain is bearing keratan sulphate chains and when said G2 domain is expressed in recombinant form and hence lacks keratan sulphate chains.

The described method may be conducted as a sandwich immunoassay using a said immunological binding partner as a capture agent or as a detection agent. As a capture agent it may be immobilised to a solid surface and as a detection agent it may suitably be labelled, e.g. with an enzyme label, a radio label or a fluorescent or other label. In such a sandwich assay, the said binding partner may be used in combination with another immunological aggrecan or aggrecan fragment binding partner as a detection or capture agent respectively. In particular, said other binding partner may be an antibody or antibody fragment having binding affinity for a neoepitope of aggrecan, being an N-terminal or a C-terminal neoepitope, possibly located in the IGD domain or between G2 and G3 or elsewhere.

Especially, the assay may be conducted as a sandwich immunoassay using a said immunological binding partner as a capture agent and using a said immunological binding partner as a detection agent. Suitably, said capture agent is provided by a said immunological binding partner immobilised on a solid surface and said detection agent is provided by a said specific binding partner bearing a detectable label.

Alternatively, an assay of the invention may be conducted as a competition immunoassay wherein (a) said immunological binding partner is immobilised to a solid surface and is incubated with said sample and a labelled competition agent comprising said G2 domain or an antibody binding portion thereof, or (b) a competition agent comprising said G2 domain or an antibody binding portion thereof is immobilised to a solid surface and is incubated with said sample and a labelled said immunological binding partner.

In an especially preferred aspect of the invention, the assay is conducted as a sandwich immunoassay using (a) a said immunological binding partner and using (b) an immunological binding partner having specific binding affinity for an N-terminal amino acid sequence comprising FFGVG . . . , either of (a) and (b) being used in said assay as a capture agent and the other of (a) and (b) being used as a detection agent.

The said immunological binding partner (b) may be the antibody AF-28 which is produced by the hybridoma cell line ATCC HB11671, previously described in the art.

Suitably, said sample is a sample of or containing synovial fluid, serum, or conditioned medium from the culture of a cartilage explant or of chondrocytes.

The invention includes an in vitro method for the detection or quantification of a marker of cartilage turnover in a sample comprising contacting the sample with an immunological binding partner which has specific binding affinity for the G2 domain of aggrecan both when said G2 domain is bearing keratan sulphate chains and when said G2 domain is expressed in recombinant form, and determining the existence or amount of specific binding of the immunological binding partner. Such a method may be carried out according to any of the procedures described above.

The sample is preferably a patient derived sample, and the method may further comprise comparing the determined level of binding with calibration values corresponding to the absence and/or the presence of a cartilage degradation disease condition.

The invention includes an immunological binding partner which has specific binding affinity for the G2 domain of aggrecan bearing keratan sulphate chains. Preferably such an immunological binding partner has specific binding affinity for the G2 domain of aggrecan, both when said G2 domain is bearing keratan sulphate chains and when said G2 domain is expressed in recombinant form. Such an immunological binding partner may be in the form of a monoclonal antibody or a fragment thereof having specific binding properties.

The invention includes hybridoma cell lines expressing monoclonal antibodies as described above and also extends to such immunological binding partners when recombinantly expressed.

The invention includes an immunoassay kit comprising an immunological binding partner of the invention, and one or more of:

a further anti-aggrecan or aggrecan fragment antibody; and an anti-aggrecan or aggrecan fragment antibody binding peptide competition agent; and optionally one or more of: a wash reagent, a buffer, a stopping reagent, an enzyme label, an enzyme label substrate, an anti-mouse antibody, calibration standards and instructions.

Generally, all previously known immunoassay formats can be used in accordance with this invention including heterogeneous and homogeneous formats, sandwich assays, competition assays, enzyme linked assays, radio-immune assays and the like.

The assays described herein are useful in the diagnosis of diseases in patients including osteoarthritis, rheumatoid arthritis and other diseases affecting cartilage tissue. In addition, the tests are useful for the assessment of disease progression, and the monitoring of response to therapy. They are also useful in exploring the production of aggrecan or aggrecan fragments in culture, e.g. culture of cartilage or of chondrocytes, and the study of the effects on such culture systems of different reagents, drug candidates and enzyme inhibitors. The immunological binding partners of the invention can be used in immunostaining of aggrecan fragment containing materials.

The term ‘immunological binding partner’ as used herein includes polyclonal and monoclonal antibodies and also specific binding fragments of antibodies such as Fab or F(ab′)₂.

Monoclonal antibodies recognising the G2-domain of aggrecan can be produced immunising mice with synthetic peptides originating from the amino acid sequence of the G2 domain, fusing the spleen-cells from selected mice to myeloma cells, and testing secreted monoclonal antibodies for binding to aggrecan. Importantly, such antibodies should also be evaluated for binding capacity to native aggrecan, e.g. by co-incubation with synovial fluid or serum samples.

Alternatively, mice could be immunised with purified, intact aggrecan and monoclonal antibodies selected for reactivity to the G2 domain. Specificity for the G2 domain could be ensured by (1) requiring reactivity with purified G2 and optionally additionally with recombinant G2 or G1-G2, or (2) by requiring reactivity with purified G1-G2 (Fosang et al., 1989) and FFGVG-containing aggrecan fragments (corresponding to MMP-cleaved aggrecan fragments containing the neoepitope FFGVG and G2) and optionally also reactivity with recombinant G2 or G1-G2; or (3) requiring reactivity with at least purified G1-G2 and lack of reactivity with recombinant G1 and synthetic IGD (the intra-globular domain separating G1 and G2.

An aspect of the invention relates to methods for detection and/or quantitation of the G2-domain of aggrecan. One such method would be a competition immunoassay using monoclonal antibodies binding to the G2 domain. Appropriately selected synthetic peptides coated onto the solid surface of a microtitre plate could compete with the sample for binding to the monoclonal antibodies. Alternatively, purified, native aggrecan could be used on the solid surface. Yet another alternative is to immobilise the monoclonal antibody on the solid surface and then co-incubate the sample with a synthetic peptide appropriately linked to a signal molecule, e.g. horseradish peroxidase or biotin.

An aspect of the present invention relates to the detection by immunoassay of the G2 domain of aggrecan in synovial fluid and serum samples.

Another aspect of the present invention relates to the detection by immunoassay of the G2 domain in the conditioned medium from cartilage explant cultures, chondrocyte cultures.

Yet another aspect of the invention relates to kits, which can be used conveniently for carrying out the methods described above. Such kits may include (1) a microtitre plate coated with synthetic peptide; (2) a monoclonal antibody recognising the G2 domain; and (3) a labelled anti-mouse IgG immunoglobulin. Alternatively, such kits may include (1) a microtitre plate coated with purified aggrecan; (2) a monoclonal antibody recognising the G2-domain; and (3) a labelled anti-mouse IgG immunoglobulin. Alternatively, such kits may include (1) a microtitre plate coated with streptavidin; (2) a synthetic peptide linked to biotin; (3) a monoclonal antibody recognising the G2 domain; and (4) a labelled anti-mouse IgG immunoglobulin. Yet another alternative could be kits including (1) a microtitre plate coated with streptavidin; (2) a synthetic peptide linked to biotin; (3) a monoclonal antibody recognising the G2-domain and conjugated to horseradish peroxidase.

A further aspect of the invention relates to the detection of aggrecan by using antibodies to unmasked epitopes in G2, in a sandwich construction.

Yet a further aspect of the invention relates to the combination of antibodies recognising the G2-domain with other antibodies recognising neo-epitopes generated during proteolytic cleavage of aggrecan. Surprisingly, combining a G2-antibody with an antibody recognising a proteolytic neo-epitope provided a major improvement in the sensitivity of the test for detection of aggrecan fragments carrying neo-epitopes.

One aspect of the invention relates to use combination of a G2-antibody with an antibody recognising a neo-epitope generated by proteolytic cleavage of aggrecan with a matrix metalloproteinase (MMP). A preferred embodiment is the use of an antibody recognising the N-terminal sequence FFGVG . . . , generated by cleavage of the intraglobular domain (IGD) between amino acid 341 and 342.

Yet another aspect of the invention relates to the use combination of a G2-antibody with an antibody recognising a neo-epitope generated by proteolytic cleavage of aggrecan with an aggrecanase. A preferred embodiment is the use of an antibody recognising the N-terminal sequence ARGS . . . generated by cleavage of the intraglobular domain (IGD) between amino acids 373 and 374. Suitable antibodies include antibody BC-3 available at abcam (ab3773) and described in Hughes et al. Biochem J (1995) 305, 799-804 and also monoclonal antibody OA-1, which is described in Pratta et al. Osteoarthritis & cartilage March 2006.

A further aspect of the invention relates to the development and use of antibodies in any of the assays described above, where the antibodies recognise unmasked epitopes located in G2 and/or G1. G1 and G2 share extended amino acid sequences with homology, and therefore such antibodies are useful according to the present invention. Other features and advantages of the present invention will be apparent from the drawings and description of preferred embodiments, the following examples, and also from the appended claims.

In the accompanying drawings:

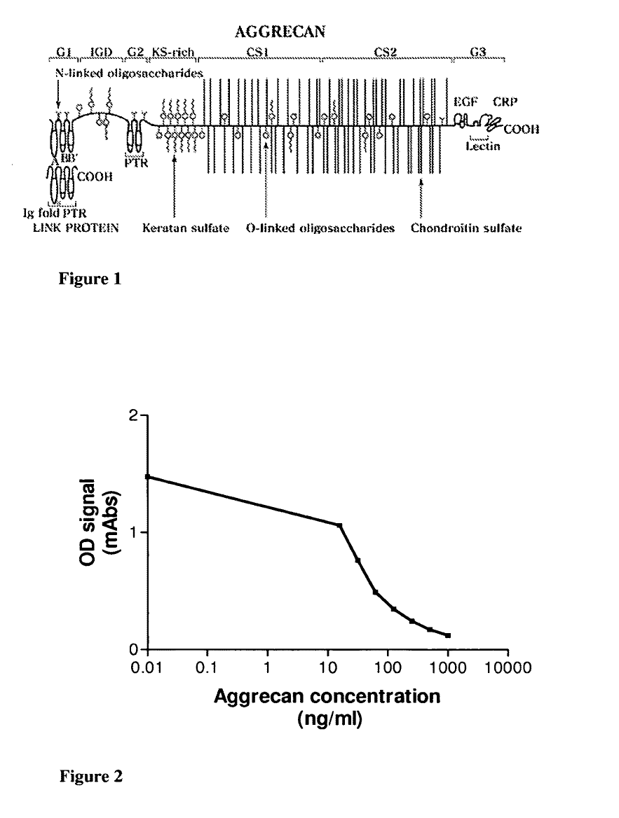

FIG. 1 shows the structure of aggrecan;

FIG. 2 shows dose dependant inhibition of binding of anti-G2 antibody F78 to aggrecan by purified porcine G1-G2;

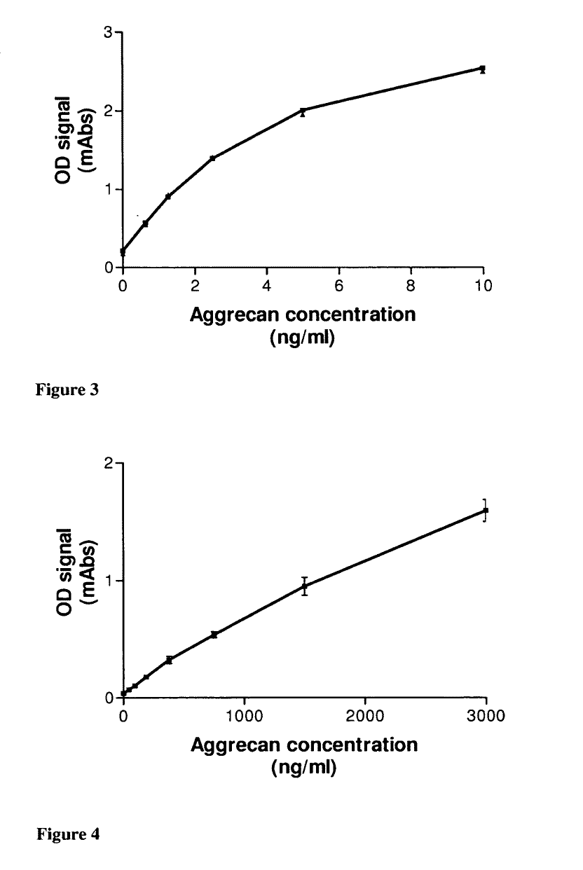

FIG. 3 shows a standard curve obtained in the immunoassay of Example 2;

FIG. 4 shows a standard curve obtained in the immunoassay of Example 3;

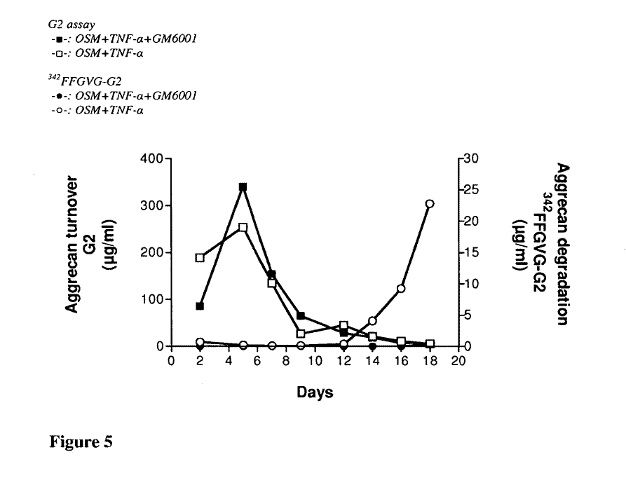

FIG. 5 shows the results obtained using a sandwich assay in Example 4;

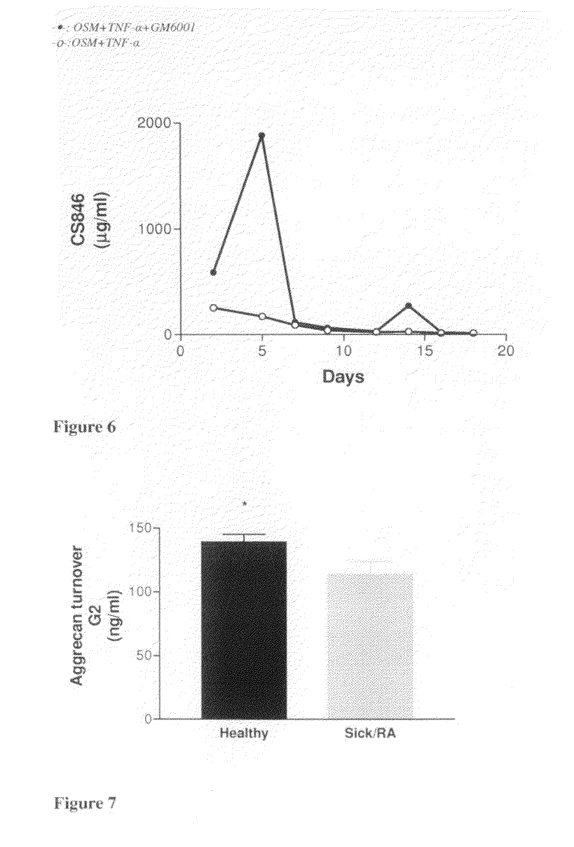

FIG. 6 shows results obtained in Example 5 using a comparative immunoassay;

FIG. 7 shows quantification results obtained using a G2 sandwich assay as described in Example 6; and

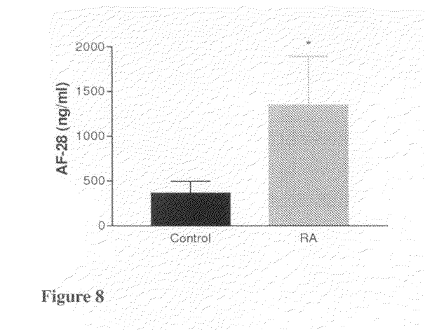

FIG. 8 shows quantification results obtained using an FFGVG-G2 sandwich assay as described in Example 7.

### EXAMPLES

### Example 1

### Generation of Monoclonal Antibodies Recognising the G2-Domain

Five seven week old female Balb/c mice were s.c. injected with intact, bovine aggrecan (SIGMA, Denmark) mixed 1:1 with Freund's Incomplete Adjuvant. The immunisation was repeated every 2 weeks for two months (five immunisations) and then continued with 4 weeks between each immunisation. Blood was obtained from the mice before immunisation initiated and one week after the fifth immunisation. The immune response was evaluated by ELISA using microtitre plates (NUNC, Denmark) coated overnight with intact, bovine aggrecan. Serial dilutions of the murine antiserum was incubated for one hour, the plates were washed and bound antibody demonstrated by incubation with sheep anti-mouse IgG antibodies conjugated to horseradish peroxidase.

When titres did not increase in the above mentioned screening test, the selected mice were rested for 4 weeks and then boosted i.p. with 200 μl immunogen without adjuvants. Three days later, the spleen was removed and used for fusion with myeloma cells using standard techniques.

Antibodies from growing hybridomas were evaluated in ELISA as described above. Hybridomas were selected for cloning by limiting dilution, propagated in culture flasks, and monoclonal antibodies were purified using Protein G affinity chromatography.

Finally, the monoclonal antibodies were selected on the basis of reactivity to un-masked epitopes located in the G2 domain (the term ‘un-masked’ here indicating epitopes which as they naturally occur are not prevented from binding by antibodies, e.g. by the presence of keratan sulphate rather than indicating epitopes from which a ‘mask’ such as keratan sulphate has been removed). This was accomplished by evaluation in a series of immunoassays. Microtitre plates were coated with intact bovine aggrecan. Binding of the monoclonal antibodies to the solid surface was determined in competition with purified, porcine G1-G2 (Fosang and Hardingham, 1989) and synthetic IGD (Chemicon, US). Monoclonal antibodies, which bound to G1-G2 but not to IGD were tested further for ability to bind to aggrecan fragments having a free C-terminus FFGVG-sequence (amino acid 342-346 in the IGD domain), which corresponds to fragments without the G1 domain (see below).

FIG. 2 demonstrates that the binding of monoclonal antibody F78 to aggrecan coated onto the solid surface of microtitre plates could be inhibited dose-dependantly by purified porcine G1-G2. In contrast, synthetic IGD (Chemicon, US) did not displace the antibody (data not shown).

### Example 2

### Sandwich Immunoassay for G2 Fragment

A sandwich assay was developed using monoclonal antibodies as described above. Streptavidin plates were incubated for 1 hour, 300 RPM, 20° C. with 100 μl 600 ng/ml biotinylated F78 monoclonal antibody against the G2 domain of aggrecan.

After incubating the plates for 1 hour, plates were washed 5 times with washing buffer (0.15 mol/l NaCl, 0.05% (v/v) Tween 20). Subsequently 100 μl standards (purified bovine aggrecan 0.05 ng/ml-10 ng/ml, or pre-diluted explant supernatants in PBS-BTB were added, and the plates were incubated for 1 hour, 300 RPM, 20° C. After the incubation period, the plates were washed 5 times as described previously, and 500 ng/ml horseradish Peroxidase (POD)-labelled F78 antibody was added. After incubating for 1 hour, 300 RPM, 20° C., the plate was washed 5 times, and 100 μl of TMB substrate was added, and the plate was incubated for 15 minutes, 300 RPM, 20° C. in the dark before 150 μl 0.18 M H₂SO₄ was added. The absorbance was measured immediately at 450 nm.

FIG. 3 shows a standard curve obtained in the abovementioned sandwich assay.

### Example 3

### Immunoassay for ³⁴²FFGVG-G2

Monoclonal antibodies to the amino acid sequence ³⁴²FFGVG has been described previously (U.S. Pat. No. 5,935,796).

Streptavidin plates were incubated for 1 hour, 300 RPM, 20° C. with 100 μl 1500 ng/ml biotinylated AF-28 monoclonal antibody against the epitope ³⁴²FFGVG-G2. Antibody dilutions were made in PBS-BTB buffer. After incubating the plates for 1 hour, plates were washed 5 times with washing buffer (0.15 mol/l NaCl, 0.05% (v/v) Tween 20). Subsequently 50 μl standards (MMP-13 digested purified porcine aggrecan 47-3000 ng/ml or pre-diluted explant supernatants in PBS-BTB were added, and the plates were incubated for 1 hour, 300 RPM, 20° C. After the incubation period, the plates were washed 5 times as described previously, and 500 ng/ml horseradish Peroxidase (POD)-labelled F78 antibody was added. After incubating for 1 hour, 300 RPM, 20° C., the plates were washed 5 times, 100 μl of TMB substrate was added, and the plates were incubated for 15 minutes, 300 RPM, 20° C. in the dark before 150 μl 0.18 M H₂SO₄ was added. The absorbance was measured immediately after at 450 nm.

FIG. 4 shows a standard curve obtained in the abovementioned assay. Importantly, the detection limit in the ³⁴²FFGVG-G2 assay was as low as 0.004 pmol/mL (corresponding to 10 ng/ml), which is considerable lower than previously reported for the competition ELISA using AF28, i.e. 7 pmol/mL (or 17500 ng/ml) (Fosang et al., 1995).

### Example 4

### Detection of G2 and ³⁴²FFGVG-G2 Fragments in Supernatants of Bovine Articular Cartilage Explants

The G2 and the ³⁴²FFGVG-G2 test described above were evaluated using conditioned medium from cartilage explants. Bovine articular cartilage was obtained from heifer stifle joints. Pieces of cartilage (16±4 mg) were placed in 96 well plates and incubated at 37° C. with 5% CO₂ and shaking (50 rpm). Serum-free D-MEM medium with or without the cytokines oncostatin M and tumour necrosis factor α (TNFα) or the MMP inhibitor GM6001 was used. As negative control, cartilage was placed in chryo-tubes, frozen in liquid N₂, and thawed at 37° C. in water-bath for three repeated freeze-thaw cycles. The explants culture medium was replaced every 3^{rd} day, and supernatants were stored at −20° C. until further analysis.

FIG. 5 shows the measurements of the aggrecan turnover in the supernatants at different days quantified by the two immuno-assays. It is seen that OSM and TNF-α stimulate an increase of ³⁴²FFGVG-G2 after day 12. When the explants are treated concomitantly in the presence of GM6001, the release of ³⁴²FFGVG-G2 is completely abrogated. Under the same experimental conditions, the release of G2 shows a completely different profile. There is an initial elevation in the release of G2 into the supernatant, but after day 5 this decreases reaching background levels after day 12. The addition of GM6001 did not inhibit the release of G2, showing that the G2 assay, different from ³⁴²FFGVG-G2, detects a fragment not dependent of MMP activity.

### Example 5

### Measurements of CS846 Release in Supernatants of Bovine Articular Cartilage Explants

The supernatants evaluated above was also measured for CS846 (IBEX CS846 competitive ELISA kit). FIG. 6 shows that OSM and TNFα do not stimulate release of CS846 into the supernatant. However, during inhibition of MMP activity a strong, initial elevation in CS846 release is observed. These data demonstrate that the analyte measured in the CS846 test is destroyed by proteolytic activity, which can be inhibited by GM6001.

### Example 6

### Detection of G2 Fragments is Lowered in RA Patients

Serum samples from 15 healthy and 15 patients with RA were quantified in the G2 assay shown in FIG. 7. The data demonstrates that the detection of released G2 molecules is significantly decreased in individuals with RA compared to controls, which means that the synthesis of aggrecan is decreased in arthritic patients.

### Example 7

### Detection of ³⁴²FFGVG-G2 Fragments is Elevated in RA Patients

The same cohorts of individuals were measured in the ³⁴²FFGVG-G2 assay as well and, in contrast to what was seen in the G2 assay, the released fragments of ³⁴²FFGVG-G2 were significantly elevated in RA patients compared to controls as seen in FIG. 8. This is in accordance with what is expected since this assay measures degradation of aggrecan.

In this specification, unless expressly otherwise indicated, the word ‘or’ is used in the sense of an operator that returns a true value when either or both of the stated conditions is met, as opposed to the operator ‘exclusive or’ which requires that only one of the conditions is met. The word ‘comprising’ is used in the sense of ‘including’ rather than in to mean ‘consisting of’.

### REFERENCES

Caterson B., Flannery C. R., Hughes C. E., Little C. B., Mechanisms involved in Cartilage Proteoglycan Catabolism. Matrix Biology 2000; Volume 19: 333-344
Fosang A. J., Hardingham T. E., Isolation of the N-terminal globular protein domains from cartilage proteoglycans, Biochemical Journal 1989; Vol 261: 801-809.
Fosang A. J., Hardingham T. E., 1-C-6 Epitope in Cartilage Proteoglycan G2 Domain is masked by Keratan Sulphate. Biochemical Journal 1991; Volume 273: 369-373
Fosang A. J., Last K., Gardiner P., Jackson D. C., Brown L., Development of a cleavage-site-specific Monoclonal Antibody for detecting metalloproteinase-derived Aggrecan Fragments in Human Synovial Fluid. Biochemical Journal 1995; Volume 310: 337-343
Fosang A. J., Last K., Maciewicz R. A., Aggrecan is degraded by matrix metalloproteinases in human arthritis. Evidence that matrix metalloproteinase and aggrecanase activities can be independent. J Clin Invest. 1996; 98 Nr. 10:2292-9
Fosang A. J., Last K., Stanton H., Weeks D. B., Campbell I. K., Hardingham T. E., Hembry R. M., Generation and novel distribution of matrix metalloproteinase-derived aggrecan fragments in porcine cartilage explants. The Journal of Biological Chemistry 2000; Volume 275 Nr. 42: 33027-33037
Garnero P., Rousseau J. C., Delmas P., Molecular Basis and Clinical use of Biochemical Markers of Bone, Cartilage and Synovium in Joint Diseases. Arthritis and Rheumatism 2000; Volume 43 No. 5: 953-968
Hardingham T. E. and Fosang A. J., The Structure of Aggrecan and its Turnover in Cartilage. The Journal of Rheumatology 1995; Volume 22:1 supplement 43: 86-89
Kongtawelert P. and Ghosh P., A new Sandwich-ELISA method for the Determination of Keratan Sulphate Peptides in Biological Fluids employing a Monoclonal Antibody and labelled avidin biotin Technique. Clinica Chimica Acta 1990; Volume 195: 17-26
Møller H. J., Larsen F. S., Ingemann-Hansen T., Poulsen J. H., ELISA for the Core Protein of the Cartilage large aggregating Proteoglycan, aggrecan: Comparison with the Concentrations of Immunogenic Keratan Sulphate in synovial fluid, Serum and Urine. Clinica Chimica Acta 1994; Volume 225: 43-55
Ratcliffe A., Shurety W., Caterson B., The quantitation of a native chondroitin sulfate epitope in synovial fluid lavages and articular cartilage from canine experimental osteoarthritis and disuse atrophy. Arthritis and Rheumatism 1993; Volume 36 No. 4:543-51

1. An immunoassay method for the detection or quantitative determination of aggrecan and/or aggrecan derived fragments in a sample comprising contacting the sample with an immunological binding partner which has specific binding affinity for the G2 domain of aggrecan bearing keratan sulphate chains, and determining the existence or amount of specific binding of the immunological binding partner.

2. A method as claimed in claim 2, wherein said immunological binding partner has specific binding affinity for the G2 domain of aggrecan, both when said G2 domain is bearing keratan sulphate chains and when said G2 domain is expressed in recombinant form.

3. A method as claimed in claim 1, conducted as a sandwich immunoassay using a said immunological binding partner as a capture agent or as a detection agent.

4. A method as claimed in claim 3, conducted as a sandwich immunoassay using a said immunological binding partner as a capture agent and using a said immunological binding partner as a detection agent.

5. A method as claimed in claim 4, wherein said capture agent is provided by a said immunological binding partner immobilised on a solid surface and said detection agent is provided by a said specific binding partner bearing a detectable label.

6. A method as claimed in claim 1, conducted as a competition immunoassay wherein (a) said immunological binding partner is immobilised to a solid surface and is incubated with said sample and a labelled competition agent comprising said G2 domain or an antibody binding portion thereof, or (b) a competition agent comprising said G2 domain or an antibody binding portion thereof is immobilised to a solid surface and is incubated with said sample and a labelled said immunological binding partner.

7. A method as claimed in claim 1, conducted as a sandwich immunoassay using (a) a said immunological binding partner and using (b) an immunological binding partner having specific binding affinity for an N-terminal amino acid sequence comprising FFGVG (SEQ ID NO:1) . . . or comprising ARGS (SEQ ID NO:4) . . . , either of (a) and (b) being used in said assay as a capture agent and the other of (a) and (b) being used as a detection agent.

8. A method as claimed in claim 6, wherein said immunological binding partner (b) is antibody AF-28 which is produced by the hybridoma cell line ATCC HB11671.

9. A method as claimed in claim 1, wherein said sample is a sample of or containing synovial fluid, serum, or conditioned medium from the culture of a cartilage explant or of chondrocytes.

10. An in vitro method for the detection or quantification of a marker of cartilage turnover in a sample comprising contacting the sample with an immunological binding partner which has specific binding affinity for the G2 domain of aggrecan both when said G2 domain is bearing keratan sulphate chains and when said G2 domain is expressed in recombinant form, and determining the existence or amount of specific binding of the immunological binding partner.

11. A method as claimed in claim 10, where the sample is a patient derived sample, further comprising comparing the determined level of binding with calibration values corresponding to the absence and/or the presence of a cartilage degradation disease condition.

12. An immunological binding partner which has specific binding affinity for the G2 domain of aggrecan bearing keratan sulphate chains.

13. An immunological binding partner as claimed in claim 12, having specific binding affinity for the G2 domain of aggrecan, both when said G2 domain is bearing keratan sulphate chains and when said G2 domain is expressed in recombinant form.

14. An immunological binding partner as claimed in claim 12, in the form of a monoclonal antibody or a fragment thereof having specific binding properties.

15. A hybridoma cell line expressing monoclonal antibodies as claimed in claim 14.

16. An immunological binding partner as claimed in claim 12, which is recombinantly expressed.

17. An immunoassay kit comprising an immunological binding partner as claimed in claim 12, and one or more of:
a further anti-aggrecan antibody; and
an anti-aggrecan antibody binding, peptide competition agent; and optionally one or more of:
a wash reagent, a buffer, a stopping reagent, an enzyme label, an enzyme label substrate, an anti-mouse antibody, calibration standards and instructions.


SEQUENCE LISTING (4 sequences)

SEQ ID NO: 1 - protein, 5 aa, Homo sapiens
FFGVG

SEQ ID NO: 2 - protein, 6 aa, Homo sapiens
ARGSVI

SEQ ID NO: 3 - protein, 6 aa, Homo sapiens
VDIPEN

SEQ ID NO: 4 - protein, 4 aa, Homo sapiens
ARGS